SEQUENCE LISTING (11 sequences)

SEQ ID NO: 1 - DNA, 16 bp, Artificial Sequence
  Oligonucleotide primer
tccagctccaatagcg

SEQ ID NO: 2 - DNA, 17 bp, Artificial Sequence
  Oligonucleotide primer
ggcatcacagacctgtt

SEQ ID NO: 3 - DNA, 20 bp, Artificial Sequence
  Oligonucleotide primer
ccgtggtcctgaaccagtta

SEQ ID NO: 4 - DNA, 20 bp, Artificial Sequence
  Oligonucldotide primer
gtcgcctgttcaccaaggat

SEQ ID NO: 5 - DNA, 46 bp, Artificial Sequence
  Oligonucleotide primer
gctcttcgtggtgtggtgtccatataaacttgtggtagttggacct

SEQ ID NO: 6 - DNA, 41 bp, Artificial Sequence
  Oligonucleotide primer
gctcttcgtggtgtggtgtcccgtccacaaaatgattctga

SEQ ID NO: 7 - DNA, 30 bp, Artificial Sequence
  Oligonucleotide primer
actgaatataaacttgtggtagttggacct

SEQ ID NO: 8 - DNA, 25 bp, Artificial Sequence
  Oligonucleotide primer
gtccacaaaatgatcctggattagc

SEQ ID NO: 9 - protein, 37 aa, Homo sapiens
MTEYKLVVVGAGGVGKSALTIQLIQNHFVDEYDPTIE

SEQ ID NO: 10 - DNA, 206 bp, Homo sapiens
ataaggcctgctgaaaatgactgaatataaacttgtggtagttggagctggtggcgtaggcaagagtgccttgacgatacagctaattcagaatcattttgtggacgaatatgatccaacaatagaggtaaatcttgttttaatatgcatattactggtgcaggaccattctttgatacagataaaggtttctctgaccattttca

SEQ ID NO: 11 - DNA, 206 bp, Homo sapiens
tgaaaatggtcagagaaacctttatctgtatcaaagaatggtcctgcaccagtaatatgcatattaaaacaagatttacctctattgttggatcatattcgtccacaaaatgattctgaattagctgtatcgtcaaggcactcttgcctacgccaccagctccaactaccacaagtttatattcagtcattttcagcaggccttat

(19) United States
(12) Patent Application Publication
(10) Pub. No.: US 2014/0335529 A1
(43) Pub. Date: Nov. 13, 2014

(54) METHOD OF TUMOR SCREENING

(71) Applicant: Philadelphia Health & Education Corporation d/b/a Drexel University College of Medicine, Philadelphia, PA (US)

(72) Inventors: YING-HSIU SU, AUDUBON, PA (US); ZHILI WANG, BRYN MAWR, PA (US); TIMOTHY M. BLOCK, DOYLESTOWN, PA (US); JANET SONG, AUDUBON, PA (US)

(73) Assignees: Philadelphia Health & Education Corporation d/b/a Drexel University College of Medicine, Philadelphia, PA (US); The Children's Hospital of Philadelphia, Philadelphia, PA (US)

(21) Appl. No.: 14/273,954

(22) Filed: May 9, 2014

Related U.S. Application Data
(63) Continuation of application No. 12/680,654, filed on Jun. 7, 2010, now abandoned.
(60) Provisional application No. 60/978,939, filed on Oct. 10, 2007.

(51) Int. Cl. C12Q 1/68

(52) U.S. Cl. CPC C12Q 1/6886; C12Q 2600/112
USPC 435/6.12

(57) ABSTRACT

A method for tumor screening using urine of a mammal, the method includes obtaining a total urine nucleic acid (e.g., DNA) from a urine sample of a mammal, extracting a high molecular weight urine nucleic acid (above 1000 bp) by contacting the total urine nucleic acid with an adsorbent in the presence of a buffer which promotes binding of the high molecular weight urine nucleic acid to the adsorbent, replacing the buffer which promotes binding of the high molecular weight urine nucleic acid with a buffer which promotes binding of the low molecular weight urine nucleic acid to the adsorbent, extracting the low molecular weight urine nucleic acid by contacting with the adsorbent, eluting the low molecular weight urine nucleic acid, and assaying the low molecular weight urine nucleic acid for a presence or absence of a gene sequence specific to a certain type of tumor.

### CROSS REFERENCE TO RELATED APPLICATIONS

This application is a continuation of U.S. Ser. No. 12/680,654, filed Jun. 7, 2010, which is the National Stage of International Application No. PCT/US2008/079505, filed Oct. 10, 2008, which claims the benefit of U.S. Provisional Application No. 60/978,939, filed Oct. 10, 2007, the entire disclosures of each of which are incorporated herein by reference.

### STATEMENT OF GOVERNMENT SUPPORT

This research was supported in part by U.S. Government funds (from the National Cancer Institute), and the U.S. Government therefore has certain rights in the invention.

### TECHNICAL FIELD

This invention relates to methods of tumor screening and detecting the presence of specific nucleic acid sequences and nucleic acid modifications by analyzing urine samples for the presence of transrenal or circulating nucleic acids.

Urinalysis for tumor DNA has been investigated for the detection of tumors located in or close to the urinary tract, such as kidney or bladder cancer (1-7). Inventors (8) and others (9;10) have shown that short length, tumor-derived DNA can be filtered through the kidney barrier and excreted into urine as illustrated in FIG. 1A. Thus, analysis of urine DNA is not to be limited to the diseases of the urinary tract and can be a valuable tool for virtually every cancer that has demonstrated DNA alterations.

As compared to collecting serum or plasma, collecting urine is a non-invasive procedure which can be done in any geographical areas and requires no special facility or equipment apart from sterile collection containers. Furthermore, the amount of urine that can be collected from a patient exceeds the amount of serum and plasma that can be collected. Thus, using urine for non-invasive cancer screening or detection is beneficial.

WO9854364A1 and related U.S. Pat. Nos. 6,287,820, 6,251,638 and 6,492,144 to Umansky et al. disclose methods of detecting and/or quantifying specific nucleic acid sequences by analyzing urine samples for nucleic acids that have crossed the kidney barrier.

U.S. Patent Publication No. US20030113758A1 to Oudet et al. discloses an in vitro method for diagnosing the predisposition of a human individual to bladder cancer or for diagnosing the occurrence of a bladder cancer in a human individual, makes use of a comparison between the allelic ratios of a serial of microsatellite markers associated with this disease, respectively in the urine DNA and in the blood cell DNA of the human individual.

U.S. Pat. No. 6,780,592 and U.S. Patent Publication No. US20050095621A1 to Sidransky disclose methods of detecting a cell proliferative disorder associated with alterations of microsatellite DNA in a sample. The microsatellite DNA can be contained within any of a variety of samples, such as urine, sputum, bile, stool, cervical tissue, saliva, tears, or cerebral spinal fluid.

It has been previously shown by inventors that urine contains DNA that resolves into two size categories (1) small (under 1,000 bp, preferably 150 bp to 250 bp) cell-free, nucleotide-sized DNA fragments (designated as low molecular weight (LMW) urine DNA) derived mostly from the circulation and (2) large (above 1000 bp) cell-associated DNA derived from the urinary tract as shown in FIG. 1B. The LMW urine DNA from the circulation is able to pass through the urinary tract and is collected in the urine, as illustrated in FIG. 1A. Inventors have demonstrated that a preferential isolation of LMW urine DNA by fractionation of total urine DNA using agarose gel electrophoresis enhanced the detection of circulating mutated K-ras DNA (8). However, the agarose gel fractionation method is very difficult and time-consuming.

Carboxylated magnetic beads have been used to purify nucleic acid from other contaminants. The size of the nucleic acids bound to carboxylated magnetic beads has been shown to vary with the binding condition (11, 12).

Thus, despite the above described efforts, there is a need for new efficient and commercially feasible methods for cancer screening and particularly, the cancer screening methods involving enhancing low MW DNA isolation from human urine.

All references cited herein are incorporated herein by reference in their entireties.

### BRIEF SUMMARY OF THE INVENTION

One aspect of the invention is a method for tumor screening using urine of a mammal, the method comprising obtaining a total urine nucleic acid (e.g., DNA) from a urine sample of a mammal, extracting a high molecular weight urine nucleic acid (above 1000 bp) by contacting the total urine nucleic acid with an adsorbent in the presence of a buffer which promotes binding of the high molecular weight urine nucleic acid to the adsorbent and thereby forming a mixture comprising a the low molecular weight urine nucleic acid, the buffer which promotes binding of the high molecular weight urine nucleic acid and optionally a trace amount of the high molecular weight urine nucleic acid, replacing the buffer which promotes binding of the high molecular weight urine nucleic acid with a buffer which promotes binding of the low molecular weight urine nucleic acid to the adsorbent, extracting the low molecular weight urine nucleic acid by contacting with the adsorbent, eluting the low molecular weight urine nucleic acid, and assaying the low molecular weight urine nucleic acid for a presence or absence of a gene sequence specific to a certain type of tumor.

In another aspect, the invention is a kit for tumor screening using urine of a mammal, the kit comprising a reagent for obtaining a total urine nucleic acid from a urine sample of a mammal; an adsorbent adapted to extract nucleic acid; a buffer which promotes binding of the high molecular weight urine nucleic acid to the adsorbent; a buffer which promotes binding of the low molecular weight urine nucleic acid to the adsorbent; an eluent for the low molecular weight urine nucleic acid; and assay materials adapted to detect a presence or absence of a gene sequence specific to a certain type of tumor in the low molecular weight urine nucleic acid.

In another aspect, the invention is a method for DNA marker screening using urine of a mammal, the method comprising obtaining a total urine nucleic acid from a urine sample of a mammal; extracting a high molecular weight urine nucleic acid having a molecular weight of at least 1000 bp by contacting the total urine nucleic acid with an adsorbent in the presence of a buffer which promotes binding of the high molecular weight urine nucleic acid to the adsorbent and thereby forming a mixture comprising a the low molecular weight urine nucleic acid having a molecular weight of below 1000 bp, the buffer which promotes binding of the high molecular weight urine nucleic acid and optionally a trace amount of the high molecular weight urine nucleic acid; replacing the buffer which promotes binding of the high molecular weight urine nucleic acid with a buffer which promotes binding of the low molecular weight urine nucleic acid to the adsorbent; extracting the low molecular weight urine nucleic acid by contacting with the adsorbent; eluting the low molecular weight urine nucleic acid from the adsorbent; and assaying the low molecular weight urine nucleic acid for a presence or absence of the DNA marker.

This invention can have multiple applications, for example, it can be used in cancer detection using nucleic acid (e.g., DNA) biomarkers (from serum, plasma, or urine), in neonatal diagnosis using circulating nucleic acid (e.g., DNA) biomarker (from serum, plasma, or urine), and in diagnostic testing for nucleic acid (e.g., DNA) biomarkers in circulation (serum, plasma, or urine).

Advantages of this invention include, enhancing the detection of circulating DNA in urine, enhancing assay sensitivity to detect DNA biomarkers that derived from the circulation using DNA isolated from urine, serum, or plasma, and enhancing assay sensitivity of cancer detection or neonatal diagnosis using circulating DNA in urine, serum, or plasma.

Human urine has been shown to resolve into two size categories: high molecular weight (MW) DNA, greater than 1 kb, from the urinary tract, and low MW DNA, between 150 to 250 bp, mostly from the circulation. Inventors developed a method to preferentially isolate low MW urine DNA by removing high MW DNA through the use of carboxylated magnetic beads. To quantify the efficacy of the removal of high MW DNA by this technique, a 18s primer (generating a PCR product of 872 bp) was designed and optimized for a real-time PCR quantification assay. Low MW DNA was isolated from the total urine DNA derived from the urine samples from 5 volunteers and then quantified by real-time PCR using the 18s primer. It was found that 92.72%±1.42% of high MW DNA was removed from the total DNA. To evaluate the effectiveness of this technique on detecting the K-ras mutation, low MW DNA was isolated from the total DNA of 40 human urine samples that had been previously tested for the K-ras mutation. Restriction Endonuclease Enriched PCR and Peptide Nucleic Acid mediated clamping real-time PCR were performed, and it was found that the detection of the K-ras mutation in the low MW DNA was much more sensitive as compared to that of the total DNA. The potential use of this finding in furthering the early detection of colorectal carcinoma is further explored.

### BRIEF DESCRIPTION OF SEVERAL VIEWS OF THE DRAWINGS

FIG. 1 (prior art) demonstrates size and nature of nucleic acid isolated from human urine. 1(A) is a schematic illustration of the possible mechanism that generates cell-free DNA in urine. 1(B) is a gel demonstrating DNA isolated from 20 ml of urine. Total urine DNA was isolated as described in Methods. DNA was aliquoted, and each aliquot was digested with RNase A, DNase I, or left untreated, and then analyzed in an 8% polyacrylamide gel. The gel was stained with ethidium bromide and photographed. The first and last lanes labeled “M” are two different DNA molecular weight markers.

FIG. 2 demonstrates removal of high MW DNA by carboxylated magnetic beads. 2(A) is a schematic representation of a procedure of the removal of high MW DNA by carboxylated beads. Magnetic beads and the magnetic holder were purchased from Agencourt Inc. 2(B) is a picture depicting evaluation of the removal of high MW DNA by gel electrophoresis. Total DNA (a mixture of 1 kb and 100 bp DNA ladder) was subjected to high MW DNA removal as illustrated in (A) with incubation times of 1 h and 2 h respectively. Three fractions, beads (B), high MW DNA (H), and low MW DNA (L) were collected, and resolved by electrophoresis in a 1% agarose gel. The gel was then stained with ethidium bromide and photographed under the UV trans-illuminator as shown.

FIG. 3 demonstrates optimization of long primers for real-time PCR quantification. 3(A) is a gel of PCR products of four sets of 18s primers. Four sets of PCR primers generating PCR product sizes ranging from 870 bp to 920 bp from the 18s gene were designed by using LC primer design software. Primers were tested using 2.5 mM MgCl₂ and an annealing temperature of 550 C to amplify 50 ng of HepG2 DNA for 40 cycles. PCR products derived from each reaction were resolved by electrophoresis in a 1% agarose gel, visualized by ethidium bromide staining, and photographed under the UV trans-illuminator. 3(B) is a PCR amplification curve of primer 18s#872 (forward primer: 5′-TCCAGCTCCAATAGCG-3′, reverse primer: 5′-GGCATCACAGACCTGTT-3′) and a linear correlation of the amplification to a 10-fold serial dilution DNA standards. A series of 10-fold dilutions of HepG2 DNA ranging from 15 ng to 0.01 5 ng were subjected to PCR amplification with primers specific to the 18s gene.

FIG. 4 relates to wild-type and mutant K-ras sequences detected in human urine and disease tissue by restriction enriched polymerase chain reactions (RE-PCR). 4(A) (prior art) is a scheme depicting a method for RE-PCR: 2 PCR and 2 BstNI digestions were performed as described in Methods and then resolved on a 9% polyacrylamide gel. The appearance of the 71 bp fragment indicates a K-ras mutation. 4(B) (prior art) is a picture of a gel demonstrating the results of an analysis on the controls for the detection of mutated K-ras sequences. This showed that 150, 15, and 1.5 copies of SW480, the positive control, decreased in their sensitivity as the number of copies decreased. HepG2, the negative control, did not show the 71 base pair fragment, and nothing was detected for H20 as expected. The sensitivity and specificity of the assay was controlled by analysis of the reconstruction standards (1.5, 15, and 150 copies of SW480 genome per 50 ng of HepG2 DNA), 50 ng HepG2 DNA (negative control), and 5 ng SW480 (positive control), as indicated. 4(C) is a picture demonstrating detection of mutated K-ras DNA in urine samples pursuant to the invention. The blinded urine specimens were subjected to total urine DNA isolation. Half of the total urine DNA was then subjected to removal of high MW DNA as the low MW DNA fraction. Total and low MW DNA were then subjected to the RE-PCR assay for mutated K-ras DNA. The photos shown represent the difference in the outcome of RE-PCR between total and low MW DNA for the same individuals as indicated in Table 2.

FIG. 5A (prior art) is a schematic representation of detection of K-ras codon 12 mutation in urine of individuals of colorectal cancer by PNA-mediated clamping PCR. FIG. 5A depicts locations and sequences of PNA nucleotides, primers and probes used in Peptide nucleic acid (PNA)-mediated clamping PCR for mutated K-ras DNA assay. FIG. 5B is a computer generated graph demonstrating detection of mutant and wild-type alleles. Reconstituted mixture of wild-type K-ras (50 ng HepG2 DNA) with 1.5, 15, and 150 genome equivalents of mutant K-ras (DNA isolated from human adenocarcinoma SW480 cells). Left and right peaks correspond to mutant and wild-type alleles, respectively. Detection of mutated K-ras DNA in total and low MW urine DNA fraction. The DNA samples containing more than 2 copies of mutated DNA per μl were subjected to PNA mediated clamping PCR assay. The melting temperature is shown.

FIG. 6 illustrates real-time PCR amplification of the 18s primer set #872. Serial dilutions of human genomic DNA (as indicated) were subjected to real-time PCR amplification with the 18s primer #872, using LightCycler FastStart DNA Master SYBR Green kit (Roche Diagnostics) according to the manufacturer's specifications (except for the concentration of MgCl2, which was 3 mM). The time and temperature in each step of the real-time PCR were 94° C. (10 sec), 55° C. (5 sec), and 72° C. (20 sec) for 45 cycles. The linear regression fit was plotted.

### DETAILED DESCRIPTION OF PREFERRED EMBODIMENTS OF THE INVENTION

The invention is based on the discovery that removing a high MW DNA fraction from a total DNA fraction in urine sample using an adsorbent such as, for example, carboxylated magnetic beads, can enhance sensitivity of a screening assay for detecting DNA markers in transrenal or circulating nucleic acids found in a low molecular weight urine DNA.

The invention is based on the discovery that an adsorbent such as, for example, carboxylated magnetic beads, can be adapted to remove high MW DNA from total urine DNA to generate a low MW urine DNA fraction, and thus enhance sensitivity of a cancer screening assay to detect DNA markers from the circulation.

The present invention provides a method for a DNA marker screening using urine of a mammal, the method comprising obtaining a total urine nucleic acid (e.g., DNA) from a urine sample of a mammal, extracting a high molecular weight urine nucleic acid by contacting the total urine nucleic acid with an adsorbent in the presence of a buffer which promotes binding of the high molecular weight urine nucleic acid to the adsorbent and thereby forming a mixture comprising a the low molecular weight urine nucleic acid, the buffer which promotes binding of the high molecular weight urine nucleic acid and optionally a trace amount of the high molecular weight urine nucleic acid, replacing the buffer which promotes binding of the high molecular weight urine nucleic acid with a buffer which promotes binding of the low molecular weight urine nucleic acid to the adsorbent, and extracting the low molecular weight urine nucleic acid by contacting with the adsorbent, eluting the low molecular weight urine nucleic acid from the adsorbent, and assaying the low molecular weight urine nucleic acid for a presence or absence of a gene sequence specific to a certain type of tumor.

In a preferred embodiment, nucleic acid is DNA.

Preferably, the adsorbent is magnetic beads treated to contain functional groups, for example, carboxylated magnetic beads.

The term “an adsorbent” includes any matrix of various shapes and forms which can selectively retain nucleic acids based on specific binding, ionic or covalent interactions and which can selectively release the retained nucleic acids optionally upon treatment with an agent which facilitates such release or removal a condition which promoted binding; such matrix can be magnetizable and includes, for example, particles described in U.S. Patent Publication US20040197780A1 to McKernan, an article by Stemmer et al., Clinical Chemistry 49: 1953-1955, 2003 and commercially available particles (e.g., magnetic particles available from Agencourt Inc. and King Fisher (e.g., silicate magnetic beads)).

The term “assaying for a presence or absence of a gene sequence specific to a certain type of tumor” includes any method of detecting mutation, for example RE-PCR and PNA-PCR.

The term “a gene sequence specific to a certain type of tumor” as used herein means nucleic acid markers (e.g., DNA markers) associated with certain tumors, for example, k-ras codon 12 mutation, p53, APC, microsatellite DNA, hypermethylation of tumor suppressor such as MGMT, GSTP-I, p 16, and MLH-1.

The term “high molecular weight urine nucleic acid” refers to nucleic acids having molecular weight equal or above 1000 bp.

The term “low molecular weight urine nucleic acid” refers to nucleic acids having molecular weight below 1000 bp.

In yet another aspect, the invention relates to a kit for tumor screening using urine of a mammal, the kit comprising a reagent for obtaining a total urine nucleic acid from a urine sample of a mammal; an adsorbent adapted to extract nucleic acid; a buffer which promotes binding of the high molecular weight urine nucleic acid to the adsorbent; a buffer which promotes binding of the low molecular weight urine nucleic acid to the adsorbent; an eluent for the low molecular weight urine nucleic acid; and assay materials adapted to detect a presence or absence of a gene sequence specific to a certain type of tumor in the low molecular weight urine nucleic acid.

In yet another aspect, the invention relates to a research tool for amplifying a nucleic acid sequence by using a forward primer: 5′-TCCAGCTCCAATAGCG-3′ and a reverse primer: 5′-GGCATCACAGACCTGTT-3′.

Materials and Methods

Study Subjects and Selection:

Participants were enrolled from the surgical or oncological services prior to initiation of chemo- or radiation therapy or surgery.

Urine Collection:

Freshly collected urine was immediately mixed with 0.5 M EDTA, pH 8.0 to a final concentration of 10 mM EDTA in order to inhibit the possible nuclease activity in the urine sample; this was stored at −70° C. To isolate total urine DNA, frozen urine samples were thawed at room temperature, and then placed immediately in ice prior to DNA isolation. Thawed urine was processed for DNA isolation within an hour.

DNA Isolation

Urine samples were mixed with 1.5 volume of 6M guanidine thiocyanate by inverting 8 times. 1 ml of resin (Wizard Plus Mini-prep DNA Purification System, Promega, Madison, Wis.) was added into the urine lysate and incubated overnight at room temperature with gentle mixing. The resin-DNA complex was centrifuged, transferred to a minicolumn (provided from the kit), and washed with a buffer provided by the manufacturer. Then, the DNA was eluted with H₂0.

DNA Quantification

Total DNA was quantified by real-time PCR using the LightCycler-Faststart DNA master SYBR Green kit (Roche, Biochemical, Germany) according to the manufacturer's specification, with primers to specifically amplify the DNA fragments containing the albumin gene (forward, 5′-CCG TGG TCC TGA ACC AGT TA-3; reverse, 5′-GTC GCC TGT TCA CCA AGG AT-3′) at an annealing temperature of 55° C. As calibrators for quantification, serially diluted genomic DNA was used.

K-Ras Codon 12 Mutation Assay

Two methods with different assay sensitivity, Restriction Enriched polymerase chain reaction (RE-PCR) and Peptide nucleic acid (PNA)-mediated clamped PCR assay, which are described previously (8; 27), were used to assay for mutated K-ras DNA.

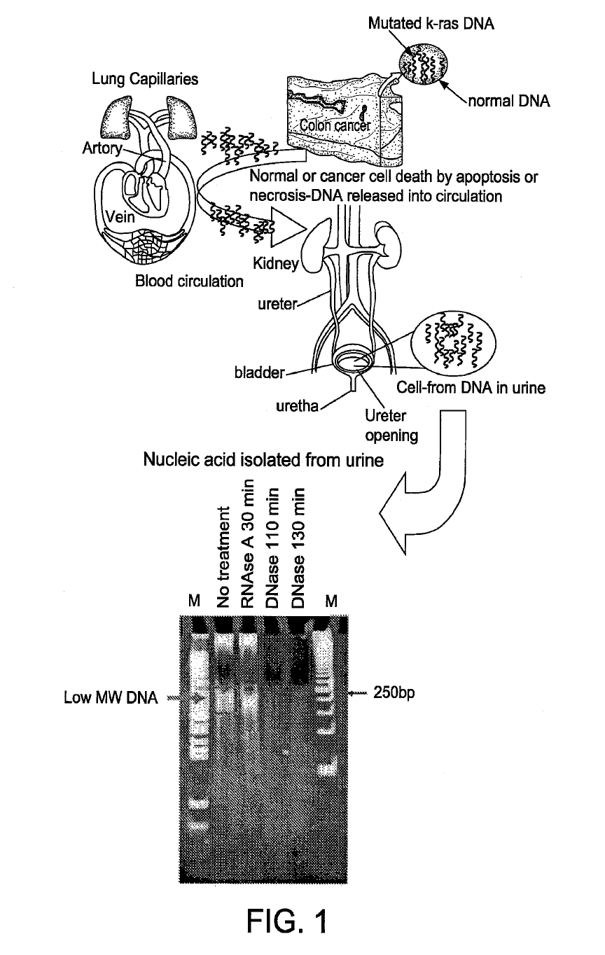

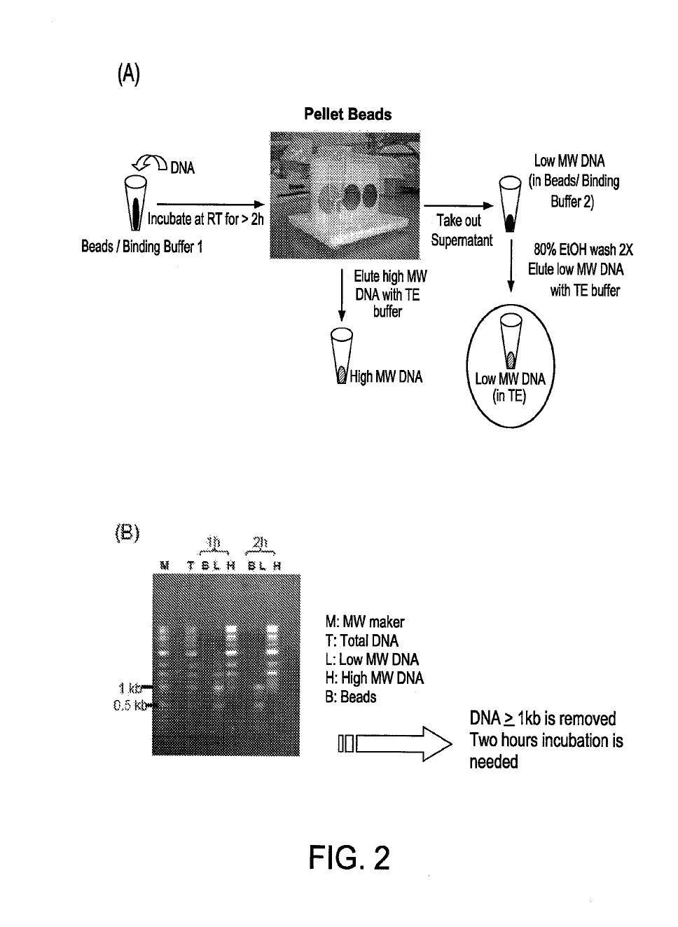

As illustrated in FIG. 2A, the RE-PCR assay is described here briefly. 20 cycles of Hot-start PCR were performed: DNA was amplified with 0.1 mM primers: L-Ext (5′ GCT CTT CGT GGT GTG GTG TCC ATA TAA ACT TGT GGT AGT TGG ACC T 3′) and R-Ext (5′ GCT CTT CGT GGT GTG GTG TCC CGT CCA CAA AAT GAT TCT GA 3′) for 20 cycles. The first 20 cycles of PCR were used to amplify both wild-type and mutated DNA in order to introduce an artificial BstNI site to the 5′ end of the amplified product derived from wild-type DNA. After the first round of PCR, the amplified products were digested with BstNI to eliminate the amplified products derived from wild-type DNA. 1/20 of the digested product was then subjected to the second Hot-start PCR of 40 cycles with the 1 mM primers L-Bst (5′ ACT GAA TAT AAA CTT GTG GTA GTT GGA CCT 3′) and R-Bst (5′ GTC CAC AAA ATG ATC CTG GAT TAG C 3′). This 2nd set of primers introduced a BstNI site to the 3′ end of amplified product derived from both wild-type and mutated templates and served as the internal control for the BstNI diagnostic digestion after the 2nd PCR. The amplified products (87 bp) of the 2nd PCR were digested to completion with BstNI (as shown by the disappearance of the 87 bp DNA fragment) and resolved through 9% polyacrylamide gels. The appearance of the 71 bp DNA fragment after BstNI digestion (as illustrated in FIG. 1(A)) is the evidence of the existence of the K-ras mutated DNA. The samples were scored as “positive” for the K-ras mutation when the 71 bp DNA fragments appeared after the 2nd BstNI digestion of the PCR products. As validation controls, the standard reconstitution samples (as in FIG. 2B) were included in each assay.

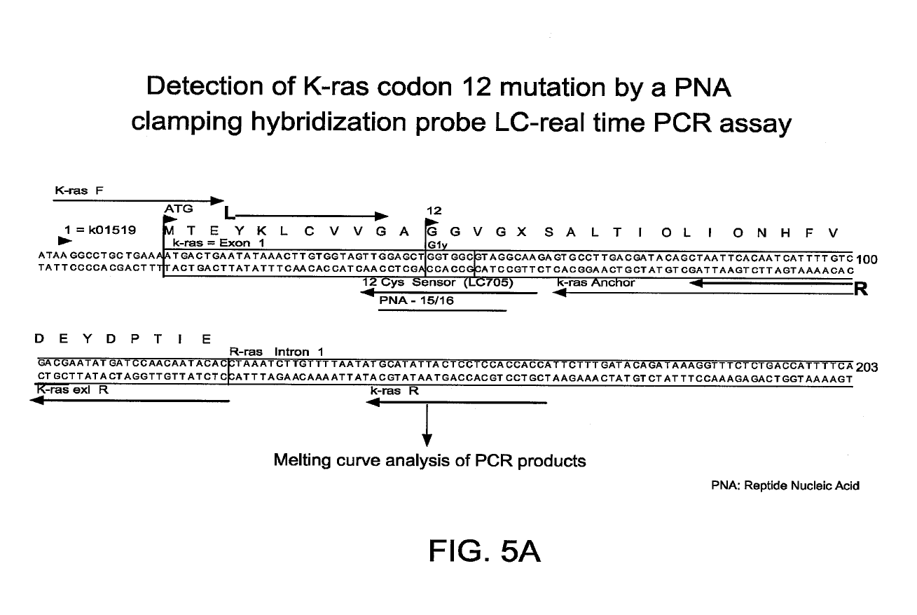

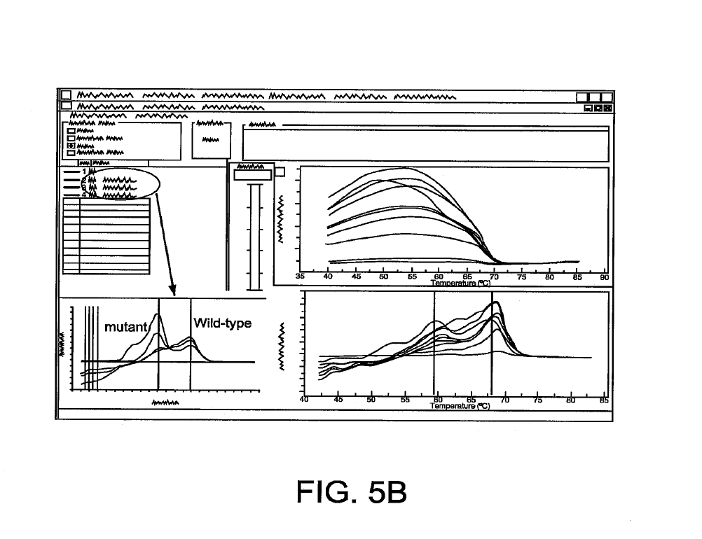

PNA-PCR is illustrated briefly in FIG. 5A, where the procedure and the locations and sequences of PNA nucleotides, primers, and probes used are listed. The PNA was designed to be the sequence of the wild-type, and thus inhibit the amplification of the wild-type template. However, due to a PNA one base pair mismatch with the mutated sequences, PCR amplification proceeded with the mutated templates. At the end of PCR, the melting temperature of each amplified product was analyzed. As validation controls, K-ras wild-type HepG2 DNA reconstituted with various amounts of K-ras codon 12 mutated SW480 DNA, the standard, was included in each assay.

The carboxylated magnetic beads were able to efficiently fractionate high MW (≧1 kb) DNA and low MW (<1 kb) DNA. Two hours incubation at room temperature was shown to obtain a more efficient fractionation as compared to one hour of incubation.

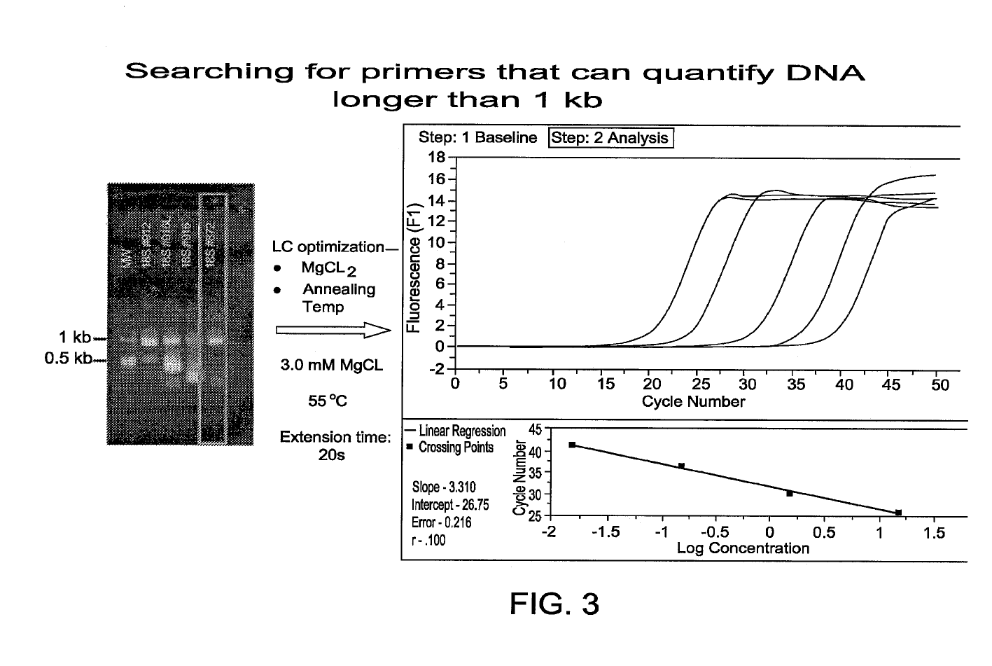

Out of the 4 primer sets that were tested, 18s#872 (forward primer: 5′-TCCAGCTCCAATAGCG-3′, reverse primer: 5′-GGCATCACAGACCTGTT-3′) was chosen for LightCycler real-time PCR optimization because it generated the most specific band as shown in FIG. 3A. To optimize real-time PCR for a long PCR product (872 bp), the extension time was increased from 10 seconds to 20 seconds with various MgCl₂ concentrations. As shown in FIG. 3B, the four ten-fold dilutions of HepG2 DNA were amplified in a linear Attorney Docket No. D2027120 174 correlation. Thus, a primer suitable to quantify only the DNA larger than 900 bp was successfully established using 3.0 mM of MgCl₂, an annealing temperature of 550 C, and an extension time of 20 seconds. The 18s#872 primer at this particular condition was then used in the following experiments to quantify DNA larger than 1 kb in size.

Determination of the Efficacy of High MW DNA Removal

To evaluate the efficacy of high MW DNA removal from total urine DNA by carboxylated magnetic beads, total urine DNA was prepared from 30 ml of urine collected from 5 individuals. Half of the total urine DNA was subjected to high and low MW DNA fractionation as illustrated in FIG. 2A. The amount of high MW DNA of each fraction (total, high and low) was then quantified by real-time PCR using primer 18s#872 as described in FIG. 2B. The percent of high MW DNA removal was calculated.

TABLE 1
Determination of the Efficacy of High MW DNA Removal

| | | | | | | Ave +/− |
|---|---|---|---|---|---|---|
| (ng/ml urine) | A | B | C | D | E | SEM |
| Total | 133.5 | 119.7 | 109.4 | 25.3 | 119.9 | — |
| Low | 10.3 | 9.8 | 4.5 | 3.0 | 5.5 | — |
| High | 119.3 | 122.7 | 85.2 | 23.2 | 71.9 | — |
| Percent of high | 92.3% | 91.8% | 96% | 88.1% | 95.4% | 92.72% ± |
| MW DNA | | | | | | 1.42 |
| removal | | | | | | |

% of high MW DNA removal = (total − low)/total × 100%
DNA conc. was quantified by using 18 s primer set (872 bp)

The percent of high MW DNA removal from the total urine DNA isolated from five individuals ranges from 88.1% to 96% with an average of 92.72%±1.42%. Thus, the carboxylated magnetic beads method that was developed can effectively remove high MW DNA from total urine DNA.

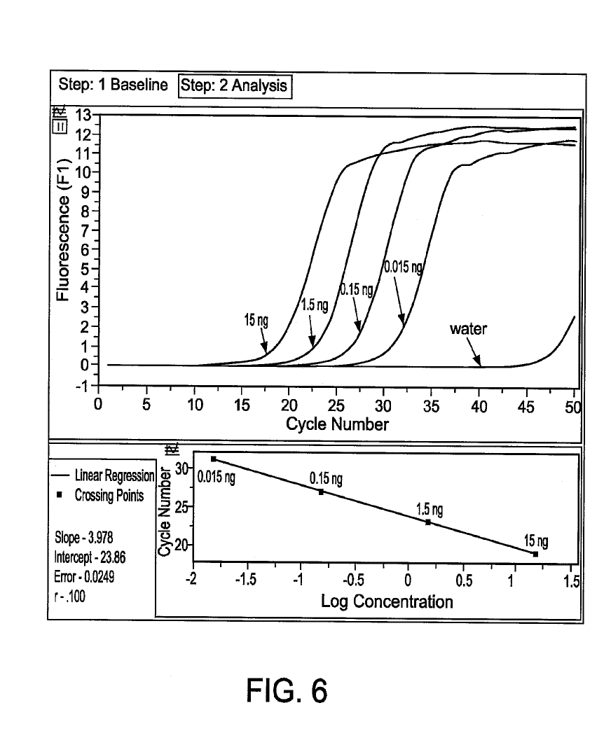

To further quantitatively measure the efficacy of high-MW DNA removal using this method, a real-time polymerase chain reaction (PCR) assay was devised. The primer set 18s#872 was employed, as described supra, and the linearity of the amplification of serial dilutions of the human control DNA by this primer set is shown in FIG. 6.

Inventors have discovered that removing high MW urine and using low MW urine DNA in a cancer detection assay improves the sensitivity of detection of mutated K-ras DNA in colorectal disease patient.

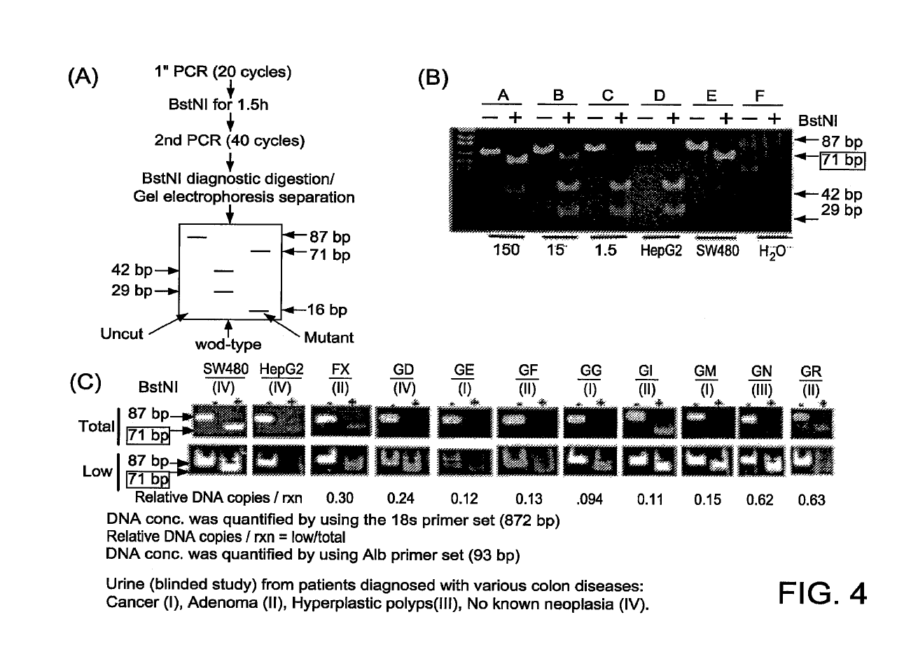

To test whether the removal of high MW DNA from total urine DNA will improve the sensitivity to detect mutated K-ras DNA, total urine DNA obtained from 40 blinded urine specimens were prepared as described in Methods. Half of the total urine DNA of each sample was subjected to the procedure (as illustrated in FIG. 2A) for the removal of high MW DNA (71 kb) and the low MW DNA fraction. Both total and low MW DNA fractions were quantified by real-time PCR for the total DNA concentration (by the Albumin primer set) and for the concentration of high MW DNA (by the 18s#872 primer set). Total and low MW DNA derived from 200 μl of urine were then subjected to the detection of mutated K-ras DNA using the RE-PCR assay as illustrated in FIG. 4A and in Table 3. The PNA-mediated PCR assay for K-ras mutation was also performed for total and low MW DNA to detect mutated K-ras as shown in FIG. 4C. The results from RE-PCR and PNA-PCR are summarized in Table 2.

TABLE 2
Detection of mutant K-ras DNA in total and low MW urine DNA of Patients with Colorectal Disease

| Code, | % of high MW DNA | | Mutant K-ras | | Code, | % of high MW DNA | | Mutant K-ras | |
|---|---|---|---|---|---|---|---|---|---|
| Diagnosis | removal | | RE-PCR | PNA | Diagnosis | removal | | RE-PCR | PNA |
| FX | Total | 0.0% | + | − | GN | Total | 0.0% | − | − |
| Adn polyps | Low | 83.0% | + | + | Hypl polyps | Low | 75.1% | + | − |
| FY | Total | 0.0% | − | − | GO | Total | 0.0% | − | − |
| Nkn | Low | 88.0% | − | − | Adn polyps | Low | 82.3% | − | − |
| FZ | Total | 0.0% | − | − | GP | Total | 0.0% | − | − |
| Adn polyps | Low | 87.6% | − | − | Adn polyps | Low | 51.9% | + | − |
| GA | Total | 0.0% | − | − | GQ | Total | 0.0% | − | − |
| CRC | Low | 97.0% | + | − | Nkn | Low | 89.1% | − | − |
| GC | Total | 0.0% | + | − | GR | Total | 0.0% | + | + |
| CRC | Low | 87.0% | + | − | Adn polyps | Low | 71.4% | + | + |
| GD | Total | 0.0% | − | − | GS | Total | 0.0% | − | − |
| Nkn | Low | 79.1% | + | + | CRC | Low | 78.7% | + | − |
| GE | Total | 0.0% | − | − | GT | Total | 0.0% | − | − |
| CRC | Low | 42.7% | + | − | CRC | Low | 71.7% | − | − |
| GF | Total | 0.0% | − | − | GU | Total | 0.0% | − | − |
| Adn polyps | Low | 90.3% | + | − | CRC | Low | 99.8% | − | − |
| GG | Total | 0.0% | − | − | GV | Total | 0.0% | − | − |
| CRC | Low | 66.9% | + | − | Adn polyps | Low | 62.8% | − | − |
| GH | Total | 0.0% | + | − | GW | Total | 0.0% | − | − |
| CRC | Low | 72.7% | + | − | Nkn | Low | 94.5% | − | − |
| GI | Total | 0.0% | + | + | GX | Total | 0.0% | + | + |
| CRC | Low | 97.5% | + | + | CRC | Low | 84.1% | + | − |
| GK | Total | 0.0% | + | − | GY | Total | 0.0% | − | − |
| CRC | Low | 92.6% | + | − | CRC | Low | 80.8% | + | − |
| GL | Total | 0.0% | − | − | HA | Total | 0.0% | − | − |
| Nkn | Low | 90.5% | − | − | Adn polyps | Low | 83.7% | + | − |
| GM | Total | 0.0% | − | − | HB | Total | 0.0% | − | − |
| Adn polyps | Low | 74.5% | + | − | Adn polyps | Low | 79.0% | + | − |
| HG | Total | 0.0% | − | − | HC | Total | 0.0% | − | − |
| (NA) | Low | 74.3% | − | − | Adn polyps | Low | 40.0% | + | − |
| HI | Total | 0.0% | − | − | HD | Total | 0.0% | + | − |
| Nkn | Low | 73.8% | − | − | CRC | Low | 42.0% | + | − |
| HK | Total | 0.0% | + | − | HE | Total | 0.0% | + | − |
| CRC | Low | 63.1% | − | − | CRC | Low | 85.5% | + | − |
| HN | Total | 0.0% | + | − | HF | Total | 0.0% | − | − |
| Adn polyps | Low | 61.3% | − | − | Nkn | Low | 76.1% | − | − |

Each urine palient sample was given a unique code to ensure a blinded study for patient diagnosis. Diagnosis of each patient was unblinded after the K-ras assays were performed. Patients were diagnosed with colorectal cancer (CRC), adenomatous polyps (Adn polyps), hyperplastic polyps (Hypl polyps), or no known neoplasia (Nkn). Diagnosis was not determined (NA) for one subject. DNA isolated from the urine of each patient was subjected to the CMB method to obtain the low MW urine DNA fraction and was quantified for high MW DNA using the 18s#872 long primer with real-time PCR. The percent of high MW DNA removal was calculated as (the amount of high MW DNA in the total urine DNA (Total) − the amount of high MW DNA in the low MW urine DNA (Low))/Total × 100%. Both total and low MW urine DNA were subjected to RE-PCR and PNA-mediated clamped PCR (PNA) assays to determine the presence (+) or absence (−) of mutant K-ras DNA. The level of detection of mutant K-ras DNA by RE-PCR is 2 copies per reaction, and the level of detection of mutant K-ras DNA by PNA is 15 copies per reaction.

TABLE 3
Analysis of mutated K-ras DNA detected by RE-PCR assay in
total and low MW urine DNA in each colorectal disease
(a blinded study of urine from 40 patients).

| | Colorectal | | Hyperplastic | No known |
|---|---|---|---|---|
| | Cancer | Adenoma | polyps | neoplasia |
| Total | 2/17 (11.8%) | 2/12 (16.7%) | 0/3 (0%) | 1/11 (9.1%) |
| Urine | | | | |
| DNA | | | | |
| Low | 9/17 (53.0%) | 7/12 (58.3%) | 1/3 (33.3%) | 2/11 (18.1%) |
| MW | | | | |
| urine | | | | |
| DNA | | | | |

The detection of the mutated K-ras sequence was more sensitive using the low MW urine DNA fragment than using the total urine DNA, although the relative number of DNA copies was much lower in the low molecular weight DNA as compared to the total urine DNA.

The frequency of detecting mutated K-ras DNA increased when low MW DNA was used in both RE-PCR and PNA clamping real-time PCR assays.

In the present invention, a method of removal of high molecular weight (MW) urine DNA by carboxylated magnetic beads was developed.

A primer suitable to quantify only the DNA larger than 900 bp was successfully established. The efficacy of high MW DNA removal from total urine DNA by carboxylated magnetic beads was evaluated as 92.72%±1.42% by real-time PCR assay.

Detection of mutated K-ras DNA in the 40 blinded urine samples from colorectal disease patients suggested that the mutated K-ras DNA was more detectable using the low MW DNA fraction as compared to the total urine DNA.

The data supports the hypothesis that carboxylated magnetic beads can be adapted to remove high MW DNA from total urine DNA to generate a low MW urine DNA fraction, and thus enhance the assay sensitivity to detect DNA markers from the circulation. The detection of mutated K-ras DNA in the low MW DNA and total urine DNA of 36 patients in another blinded urine study was compared and is summarized in Table 4. The participants either had colorectal cancer (n=16), adenomatous polyps (n=12), hyperplastic polyps (n=2), or no known neoplasia (n=7). For the colorectal cancer group, the K-ras mutations were detected in 43.8% of total urine DNA samples as compared to 87.5% of low MW urine DNA samples (p-value=0.013 by Fisher's exact test). For the adenomatous polyps group, the K-ras mutations were detected in 16.7% of total urine DNA samples in contrast to 75% of low MW urine DNA samples (p-value=0.005 by Fisher's exact test). The detection of K-ras mutations in total and low MW urine DNA was not compared in subjects with hyperplastic polyps or no known neoplasia due to an insufficient sample size and lack of available tissue DNA respectively.

The concordance between the detection of K-ras codon 12 mutations in disease tissue and its corresponding total and low MW urine DNA was determined (Table 4). It was shown that the low MW urine DNA fraction (86% concordance) had a significantly better concordance with tissue DNA than total urine DNA (38%) with a p-value of 0.0015 by the chi-square test. This strongly indicates that the use of low MW urine DNA fraction isolated using the developed CMB method is more sensitive than use of total urine DNA in detecting the K-ras codon 12 mutations in the urine of colorectal disease patients.

TABLE 4
Analysis of mutated K-ras DNA detected by RE-PCR assay in total and low MW urine
DNA in each colorectal disease (a blinded study of urine from 40 patients).

| CRC | | | | Adn polyps | | | | Hypl polyps | | | | Nkn | | |
|---|---|---|---|---|---|---|---|---|---|---|---|---|---|---|
| (n = 16, | | | | (n = 12, | | | | (n = 2, | | | | (n = 7, | | |
| mean age = 64, | | | | mean age = 60, | | | | mean age = 51.5, | | | | mean age = 52.7, | | |
| range 33-87, 6 females) | | | | range 41-80, 5 females) | | | | range 51-52, 0 female) | | | | range 50-61, 5 females) | | |
| Code | Tiss | T | L | Code | Tiss | T | L | Code | Tiss | T | L | Code | T | L |
| GA | + | − | + | FX | NA | + | + | GJ | − | − | − | FY | − | − |
| GC | NA | + | + | FZ | NA | − | − | GN | + | − | + | GD | − | + |
| GE | NA | − | + | GF | + | − | + | | | | | GL | − | − |
| GG | + | − | + | GM | − | − | + | | | | | HI | − | − |
| GH | + | + | + | HN | NA | − | + | | | | | GQ | − | − |
| GI | − | + | + | GO | + | − | − | | | | | GW | − | − |
| GK | + | + | + | GP | + | − | + | | | | | HF | − | − |
| HK | NA | − | + | GR | + | + | + | | | | | | | |
| GS | + | − | + | GV | − | − | − | | | | | | | |
| GT | NA | − | − | HA | + | − | + | | | | | | | |
| GU | NA | − | − | HB | + | − | + | | | | | | | |
| GX | NA | + | + | HC | + | − | + | | | | | | | |
| GY | + | − | + | | | | | | | | | | | |
| GZ | + | − | + | | | | | | | | | | | |
| HD | + | + | + | | | | | | | | | | | |
| HE | + | + | + | | | | | | | | | | | |
| # tested | # “+” | # “+” | # “+” | # tested | # tested | # “+” | # “+” | # tested | # tested | # “+” | # “+” | # tested | # “+” | # “+” |
| 16 | 10 | 7 | 14 | 12 | 9 | 2 | 9 | 2 | 2 | 0 | 1 | 7 | 0 | 1 |

| | | | | | | | | |
|---|---|---|---|---|---|---|---|---|
| % pos | 43.8% | 87.5% | 16.7% | 75.0% | 0% | 50% | 0% | 14.2% |

| | | | |
|---|---|---|---|
| p-value | p = 0.0233 | p = 0.0123 | NA |

| | | |
|---|---|---|
| Concordance (p = 0.0015) | Total urine DNA: 38% (8/21) | Low MW urine DNA: 86% (18/21) |

The method of the invention can be used with a 96-well plate technology and can be further automated as know to persons skilled in the art to reduce possibility of human errors. Automation equipment for CMB, which can be used to isolate or purify up to 384 samples at once, is commercially available.

The invention will be illustrated in more detail with reference to the following Examples, but it should be understood that the present invention is not deemed to be limited thereto.

### EXAMPLES

Preferential fractionation of high/low MW DNA using the carboxylated magnetic beads.

The protocol was developed using carboxylated magnetic beads purchased from Agencourt, Inc., Beverly, Mass. This method should be suitable for the carboxylated magnetic beads from other sources as well).

To fractionate DNA sample into high MW DNA (71 kb) and low MW DNA (<1 kb) fractions, two binding buffers are used to differentially bind the DNA of interest by size on carboxylated magnetic beads. First binding buffer, high MW DNA binding buffer, is composed of 8% polyethylene glycol (PEG) 8000, 0.3 M NaCl. The second binding buffer, low MW DNA binding buffer, is composed of 1 Volume of the unbound portion from high MW DNA removal, 0.85 Volume of isopropanol, 10 μl carboxylated magnetic beads—if DNA is less than 3 μg, scale up the amount of beads used, if more DNA is expected. Thus the low MW DNA binding buffer is composed of 4.1% of PEG, 0.15 M of NaCl, 43.5% of isopropanol and beads.

The first step is to bind the high MW DNA to the beads by mixing DNA with the beads in the high MW DNA binding buffer and incubating the mixture at room temperature (RT) for 2 h with gentle rocking. The high MW DNA-beads complex is separated from the low MW DNA (in the solution) by using Agencourt APRIPlate magnetic plate. Low MW DNA (in solution) is then transferred to another tube and binds to the beads with additional isopropanol and carboxylated magnetic beads to make up to a low MW DNA binding buffer and incubating with gentle rocking at RT for 30 min. The low MW DNA-beads complex is separated from the unbound fraction.

To elute the DNA, the DNA-beads complex will be washed twice by 75% EtOH, airdried, and the DNA can be eluted in either water or 1×TE buffer.

To isolate and assay circulating DNA from total urine DNA, the low MW DNA fraction is eluted for further analysis.

While the invention has been described in detail and with reference to specific examples thereof, it will be apparent to one skilled in the art that various changes and modifications can be made therein without departing from the spirit and scope thereof.

### REFERENCES

1. Utting M, Werner W, Dahse R, Schubert J, Junker K. Microsatellite Analysis of Free Tumor DNA in Urine, Serum, and Plasma of Patients: A Minimally Invasive Method for the Detection of Bladder Cancer. Clin Cancer Res 2002; 8:35-40.
2. Hogue M O, Lee J, Begum S, Yamashita K, Engles J M, Schoenberg M et al. High-Throughput Molecular Analysis of Urine Sediment for the Detection of Bladder Cancer by High-Density Single-Nucleotide Polymorphism Array. Cancer Res 2003; 63:5723-6.
3. Cairns P. Detection of Promoter Hypermethylation of Tumor Suppressor Genes in Urine from Kidney Cancer Patients. Ann NY Acad Sci 2004; 1022:40-3.
4. Dulaimi E, de Caceres 11, Uzzo R G, A1 Saleem T, Greenberg R E, Polascik T J et al. Promoter Hypermethylation Profile of Kidney Cancer. Clin Cancer Res 2004; 10:3972-9.
5. Dulaimi E, Uzzo R G, Greenberg R E, A1 Saleem T, Cairns P. Detection of Bladder Cancer in Urine by a Tumor Suppressor Gene Hypermethylation Panel. Clin Cancer Res 2004; 10: 1887-93.
6. Cairns P, Esteller M, Herman J, Schoenberg M, Jeronimo C, Sanchez-Cespedes M et al. Molecular detection of Prostate cancer in urine by GSTP1 Hypermethylation. Clin Cancer Res 2001; 7:2727-30.
7. Papadopoulou E, Davilas E, Sotiriou V, Koliopanos A, Aggelakis F, Dardoufas K et al. Cell-free DNA and RNA in plasma as a new molecular marker for prostate cancer. Oncology Research 2004; 14:439-45.
8. Su Y-H, Wang M, Brenner D E, Ng A K, Melkonyan H, Umansky S et al. Human urine contains small, 150-250 nucleotide sized, soluble DNA derived from the circulation and may be useful in the detection of colorectal cancer. Journal of Molecular Diagnostics 2004; 6, 101-107
9. Botezatu I, Serdyuk 0, Potapova G, Shelepov V, Alechina R, Molyaka Y et al. Genetic analysis of DNA excreted in Urine: A new approach for detecting specific genomic DNA sequences from cells dying in an organism. Clin Chem 2000; 46: 1078-84.
10. Serdyuk 01, Botezatu I, Shelepov V, Potapova G, Alekhina R. P., Molyaka Y K et al. Detection of mutant k-ras sequences in the urine of cancer patients. Bull Exp Biol Med 2001; 13 1:283-4.
11. DeAngelis M, Wang D, Hawkins T. Solid-phase reversible immobilization for the isolation of PCR products. Nucl Acids Res 1995; 23:4742-3.
12. Hawkins T, O'Connor-Morin T, Roy A S C. DNA purification and isolation using a solid-phase. Nucl Acids Res 1994; 22:4543-4.
13. Su Y H, Wang M, Block T M, Landt 0, Botezatu I, Serdyuk 0 et al. Transrenal DNA as a Diagnostic Tool: Important Technical Notes. Ann NY Acad Sci 2004; 1022:81-9.

What is claimed:

1. A method for tumor screening using urine of a mammal, the method comprising:
obtaining a total urine nucleic acid from a urine sample of a mammal, the total urine nucleic acid comprising a high molecular weight urine nucleic acid having a molecular weight of at least 1000 bp and a low molecular weight urine nucleic acid having a molecular weight of below 1000 bp;
extracting the high molecular weight urine nucleic acid by contacting the total urine nucleic acid with an adsorbent in the presence of a buffer which promotes binding of the high molecular weight urine nucleic acid to the adsorbent and thereby forming a mixture comprising the low molecular weight urine nucleic acid the buffer which promotes binding of the high molecular weight urine nucleic acid, and optionally a trace amount of the high molecular weight urine nucleic acid;
replacing the buffer which promotes binding of the high molecular weight urine nucleic acid with a buffer having a salt concentration of no more than 0.15 M, which promotes binding of the low molecular weight urine nucleic acid to the adsorbent;
extracting the low molecular weight urine nucleic acid by contacting with the adsorbent;
eluting the low molecular weight urine nucleic acid from the adsorbent; and
assaying the low molecular weight urine nucleic acid for a presence or absence of a gene sequence specific to a certain type of tumor.

2. The method of claim 1, wherein the total urine nucleic acid is a total urine DNA, the high molecular weight urine nucleic acid is a high molecular weight urine DNA, and the low molecular weight urine nucleic acid is a low molecular weight urine DNA.

3. The method of claim 1, wherein the low molecular weight urine nucleic acid is 400 bp or less.

4. The method of claim 1, wherein the adsorbent is carboxylated magnetic beads.

5. The method of claim 1, wherein said assaying is performed by at least one of RE-PCR or PNA-PCR.

6. A kit for tumor screening using urine of a mammal, the kit comprising:
a reagent for obtaining a total urine nucleic acid from a urine sample of a mammal, the total urine nucleic acid comprising a high molecular weight urine nucleic acid and a low molecular weight urine nucleic acid;
an adsorbent adapted to extract nucleic acid;
a buffer which promotes binding of the high molecular weight urine nucleic acid to the adsorbent;
a buffer having a salt concentration of no more than 0.15 M which promotes binding of the low molecular weight urine nucleic acid to the adsorbent;
an eluent for the low molecular weight urine nucleic acid; and
assay materials adapted to detect a presence or absence of a gene sequence specific to a certain type of tumor in the low molecular weight urine nucleic acid.

7. A method for DNA marker screening using urine of a mammal, the method comprising:
obtaining a total urine nucleic acid from a urine sample of a mammal, the total urine nucleic acid comprising a high molecular weight urine nucleic acid having a molecular weight of at least 1000 bp and a low molecular weight urine nucleic acid having a molecular weight of below 1000 bp;
extracting the high molecular weight urine nucleic acid by contacting the total urine nucleic acid with an adsorbent in the presence of a buffer which promotes binding of the high molecular weight urine nucleic acid to the adsorbent and thereby forming a mixture comprising the low molecular weight urine nucleic acid the buffer which promotes binding of the high molecular weight urine nucleic acid, and optionally a trace amount of the high molecular weight urine nucleic acid;
replacing the buffer which promotes binding of the high molecular weight urine nucleic acid with a buffer having a salt concentration of no more than 0.15 M which promotes binding of the low molecular weight urine nucleic acid to the adsorbent;
extracting the low molecular weight urine nucleic acid by contacting with the adsorbent;
eluting the low molecular weight urine nucleic acid from the adsorbent; and
assaying the low molecular weight urine nucleic acid for a presence or absence of the DNA marker.
